(19) United States
(12) Patent Application Publication
(10) Pub. No.: US 2026/0027306 A1
(43) Pub. Date: Jan. 29, 2026

(54) AUTOINJECTOR

(71) Applicant: Owen Mumford Limited, Woodstock, Oxfordshire (GB)

(72) Inventors: Robert William BRUNS, Woodstock, Oxfordshire (GB); Daniel James BYRNE, Woodstock, Oxfordshire (GB)

(21) Appl. No.: 19/120,895

(22) PCT Filed: Oct. 17, 2023
(86) PCT No.: PCT/EP2023/078889

§ 371 Date: Apr. 14, 2025

(30) Foreign Application Priority Data
Oct. 17, 2022 (GB) 2215314.2

(51) Int. Cl. A61M 5/315; A61M 5/20

(52) U.S. Cl. CPC A61M 5/31568; A61M 5/2033; A61M 5/31576; A61M 2005/31588

(57) ABSTRACT

An autoinjector comprises a main housing for receiving a syringe or cartridge, and a drive mechanism located substantially within the main housing for providing motive force. The drive mechanism comprises a plunger driver movable through the housing, between a stationary start position and a stationary end position, to apply said motive force to a plunger of the syringe or cartridge. The autoinjector further comprises a contactless proximity sensor and detection feature, one of which is fixed with respect to the main housing and the other being fixed with respect to the plunger driver. When the plunger driver is in one of the stationary start position and the stationary end position, the detection feature is located at a position within a sensing region of the contactless proximity sensor and, for substantially all of the travel of the plunger driver between the stationary start position and stationary end position, the detection feature is located outside of the sensing region of the contactless proximity sensor.

### TECHNICAL FIELD

The present invention relates to autoinjectors for drug delivery and provided with sensors for monitoring operation.

### BACKGROUND

Mechanically powered autoinjectors are commonly used to deliver many different types of drug. In the majority of injectors, the force to deliver an injection is provided by powerful helical springs, either compressed and released to provide the delivery force or expanded and contracted to provide that force. In some cases, the same or a different spring mechanism provides a needle insertion force to cause a syringe or cartridge needle tip to penetrate a user's skin, prior to drug delivery.

It is of course of the utmost importance that medication delivery regimens are followed precisely, e.g. in terms of drug delivered per injection and the timing of injections. This has traditionally relied on careful use by patients or their medical practitioners or carers. Particularly for self-injections, where users may be elderly or otherwise frail, it can be very difficult to follow a set regimen and injections may be missed or too many carried out, or the wrong dose delivered, potentially leading to serious consequences.

With the widespread use of smartphones and other wirelessly connected personal devices, interest is growing in the use of autoinjectors provided with electronic monitoring means and wireless connectivity, such as will allow the automatic monitoring of injections and reporting to user devices, possibly with further onward reporting to some central monitoring service or medical practice. Whilst such electronic monitoring and reporting functionality may be used in conjunction with electrical/electronic drive means for injection delivery (and needle insertion), at least for the time being it is considered preferable to continue using a mechanical drug delivery mechanism to ensure reliability and the possibility to perform an injection should electrical power (e.g. from a rechargeable battery) be unavailable.

WO2019086718 describes an implementation of a mechanically powered autoinjector with electronic monitoring. Detection relies upon the use of micro-switches although it is suggested that other means for detection may be used.

A potential problem with micro-switches and other previously proposed detectors is the extremely high speed at which components of the autoinjector may move. In some cases, firing of the device may result in a relatively uninhibited initial movement of a (spring driven) plunger driver through the housing prior to the driver coming into contact with a syringe plunger, whereupon the speed of the driver is significantly reduced. Such initial rapid movement occurs where the plunger driver and syringe plunger are initially separated, which could result from the syringe medicament container being partially filled (e.g., a 0.2 ml from a 1 ml syringe) for example. The initial travel of the plunger driver to the syringe plunger meets little to no resistance and so the plunger driver accelerates rapidly. Mechanically actuated switches tend to be relatively bulky which can be problematic when trying to create a compact device and may be prone to failure.

An option considered to detect the passage and/or position of the driver may be a Hall effect sensor. Such sensors might be used to detect the passage of a magnetic field generated by a magnet (or electromagnet) attached to the driver. However, the sampling frequency of a Hall effect sensor may be of the order of 20 samples per second. As such, it may not be able to detect movement of the driver, and attached magnet, at the typical operating speed of an autoinjector, at least during an initial high speed movement.

### SUMMARY

According to a first aspect of the present invention there is provided an autoinjector for delivering a dose of medication into a patient from a medication containing syringe or cartridge having a needle affixed thereto. The autoinjector comprises a main housing for receiving the syringe or cartridge, a drive mechanism located substantially within the main housing for providing motive force to deliver medication into the patient from the syringe or cartridge, the drive mechanism comprising a plunger driver axially movable through the housing, between a stationary start position and a stationary end position, to apply said motive force to a plunger of the syringe or cartridge, a contactless proximity sensor and a detection feature for detection by the contactless proximity sensor, and an electrically powered monitoring unit contained substantially within the main housing and electrically coupled to the contactless proximity sensor. One of the contactless proximity sensor and the detection feature is fixed with respect to said main housing and the other being fixed with respect to the plunger driver. When the plunger driver is in one of the stationary start position and the stationary end position, the detection feature is located at a position within a sensing region of the contactless proximity sensor and, for substantially all of the travel of the plunger driver between the stationary start position and stationary end position, the detection feature is located outside of the sensing region of the contactless proximity sensor.

The drive mechanism comprises one or more springs configured to be primed by user action and which, when primed, generate said motive force.

The autoinjector may comprise a second contactless proximity sensor fixed with respect to the main housing or the plunger driver, wherein, when the plunger driver is in the other of the stationary start position and the stationary end position, the detection feature is located at a position within a sensing region of the second contactless proximity sensor and, for substantially all of the travel of the plunger driver between the stationary start position and stationary end position, the detection feature is located outside of the sensing region of the second contactless proximity sensor.

The autoinjector may comprise a display for displaying an operating state of the autoinjector in dependence upon signals generated by the contactless proximity sensor or sensors.

The main housing may comprise a main body and a lid hingedly coupled together, wherein said drive mechanism is mounted to said main body and the or each contactless proximity sensor is fixed to the lid.

The plunger driver may comprise a push member configured in use to locate behind a rear end of a plunger of a syringe received within the main housing and to apply said motive force to the plunger, the detection feature being located within said push member.

The or each contactless proximity sensor may be a Hall effect sensor and the detection feature may be a magnetic or ferromagnetic component.

According to a second aspect of the present invention there is provided an autoinjector for delivering a dose of medication into a patient from a medication containing syringe or cartridge having a needle affixed thereto. The autoinjector comprises a main housing for receiving the syringe or cartridge, and a drive mechanism located substantially within the main housing for providing motive force to deliver medication into the patient from the syringe or cartridge, the drive mechanism comprising a plunger driver axially movable through the housing, between a stationary start position and a stationary end position, to apply said motive force to a plunger of the syringe or cartridge. The autoinjector further comprises a linear rheostat comprising a resistance element and a sliding contact, one of the resistance element and the sliding contact being fixed relative to the main housing and the other being fixed relative to the plunger driver, and a detection circuit comprising a battery and a current detector coupled in series with the rheostat and configured to provide a signal indicative of a resistance presented by the rheostat.

### BRIEF DESCRIPTION OF THE DRAWINGS

FIG. 1A-C show an autoinjector in a (A) closed, (B) partially open and (C) open state;

FIG. 2 shows a safety syringe;

FIG. 3 shows a capped end of the autoinjector of FIGS. 1A-C;

FIG. 4A and 4B show partial cross-sections of the autoinjector of FIGS. 1A-C;

FIG. 5 shows an axial cross-sectional view of a rear part of the autoinjector of FIGS. 1 to 4; and

FIG. 6 illustrates schematically a further embodiment of an autoinjector comprising a resistance slider to detect plunger driver position and/or motion.

### DETAILED DESCRIPTION

As has been noted above, it can be challenging to use a Hall effect sensor to detect the departure of a plunger driver of an autoinjector from a start position, or its arrival at an end position, due to the relatively rapid movement of the plunger driver across the sensing region of the sensor. Proposed is an autoinjector that overcomes or mitigates this problem by locating a sensed component such as a magnet or ferromagnetic component on the plunger driver or otherwise fixed relative thereto and which sits within the sensing region when in one or other of its stationary positions.

The terms “forward” or “front” are used here to refer to the needle side or injection site end of the autoinjector, whereas the term “rear” refers to the end of the autoinjector remote from the needle or injection site.

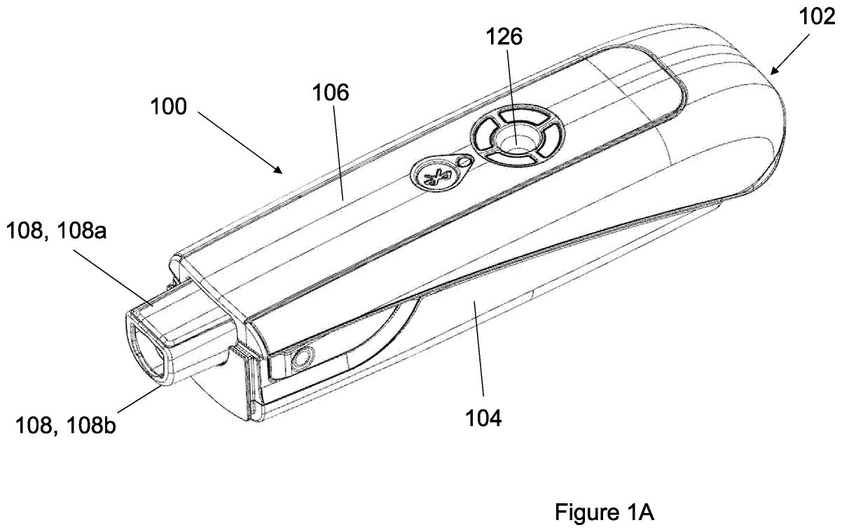

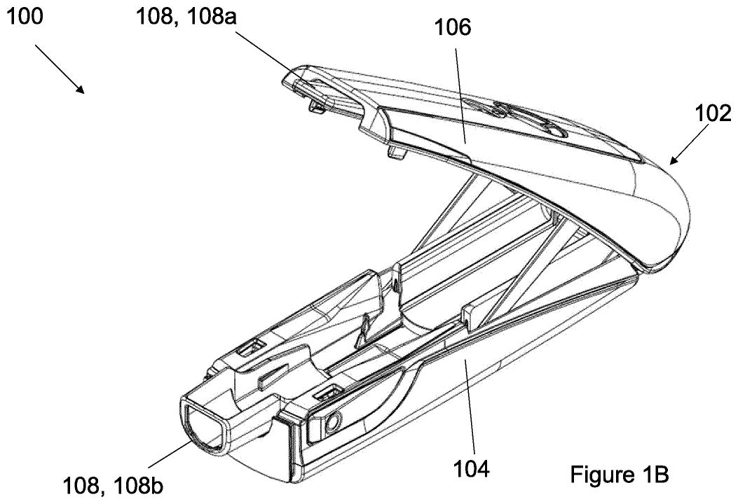

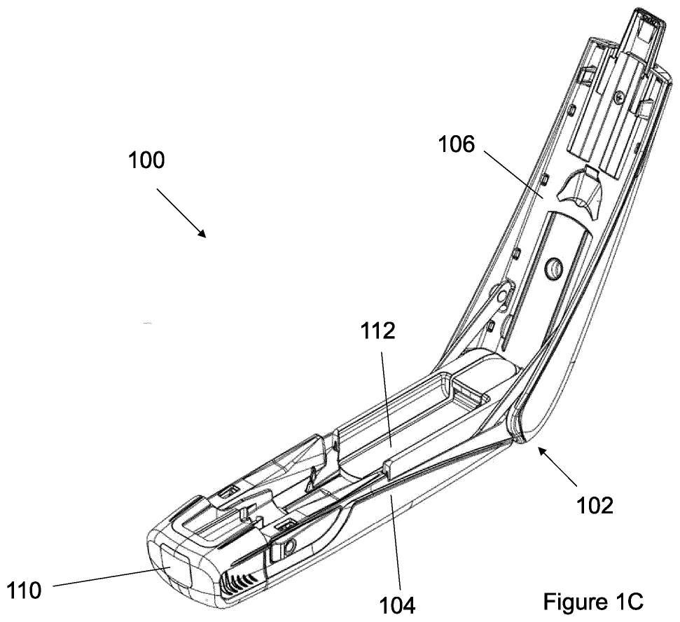

FIGS. 1A-C show an embodiment of an autoinjector 100 in: A) a closed state; B) a partially open state; and C) a fully open state.

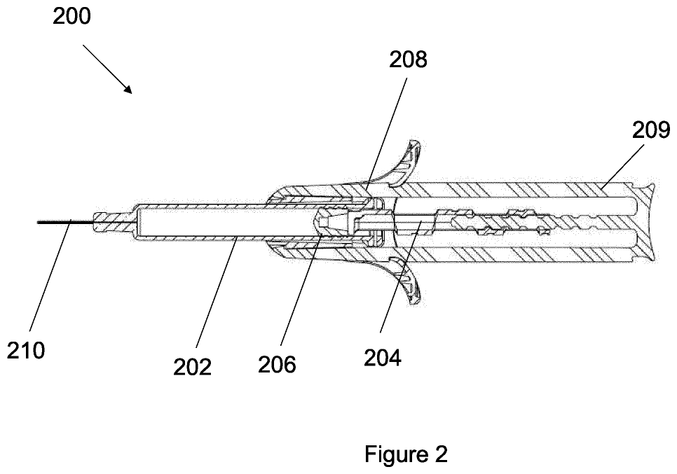

The autoinjector 100 comprises a housing 102 which includes a main body 104 and a lid 106 that are hingedly connected so as to permit opening and closing of the housing. The autoinjector further comprises a plurality of component parts contained within the housing. A syringe such as the syringe 200 of FIG. 2 (not shown in FIGS. 1A-C) is receivable within the housing in a slot 112 defined in the main body. The lid 106 of the autoinjector 100 includes a through-hole 126 positioned so that a surface of a plunger driver 116, the operation of which is described below, is viewable once firing is complete. That surface is vividly coloured in contrast to other parts visible through the through-hole prior to and during drug delivery to thereby provide a visual indication to the user that drug delivery is complete.

As shown most clearly in FIGS. 1A and 1B, the autoinjector 100 further comprises a shroud 108, formed from a lower part 108a and an upper part 108b. The lower and upper parts are respectively coupled to the main body 104 and lid 106 so that the parts 108a, 108b separate as the housing 102 is opened to allow insertion of a syringe 200 and come together to form the unitary shroud 108 when the housing is closed. The shroud 108 defines an aperture through which the needle 210 of a syringe 200 at least partially extends, when a syringe is received in the autoinjector 100. The lower part and upper part 108a, 108b comprise slidable connections with the main body 104 and lid 106 respectively, to permit movement between an extended position where the end of the syringe needle is substantially covered by the shroud, and a retracted position where the end of the syringe needle is exposed. The shroud parts are separately biased towards the extended position so that the needle of a syringe in the autoinjector remains 30 substantially covered prior to an injection.

As shown in FIG. 1C, the autoinjector 100 includes a removable cap 110 which is normally in place prior to an injection being performed. For ease of understanding, the cap is omitted from FIGS. 1A and 1B. In the configuration shown, the cap fits slidably over the lower shroud part 108b and further abuts against a front end of the main body 104. The cap 110 prevents user access to the shroud and hence accidental firing while the cap is in place.

FIG. 2 illustrates a safety syringe 200 suitable for use with the described autoinjector 100. Such a syringe 200 is described in detail in WO2019086718. It is sufficient here to note that the syringe comprises a syringe body 202 for containing a medicament, a syringe plunger 204 that engages with a bung 206 within the syringe body, a needle shield 208 coupled to a safety plunger 209, and a needle 210. The coupling between the syringe plunger and the safety plunger/needle shield is such that the needle shield 208 is deployed around the needle of the syringe so as to substantially cover the needle following delivery of the medicament from the syringe body. This coupling is described in detail in WO2019/086718.

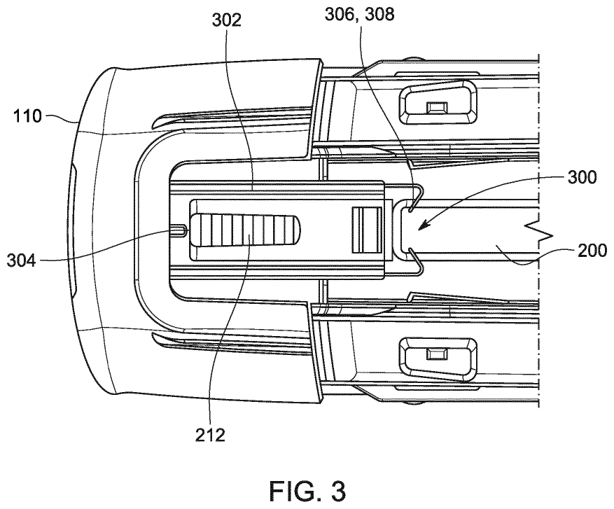

In general, syringes, including safety syringes, are routinely provided with a protective rigid needle shield (RNS) which require removal before a syringe can be used (the RNS is not shown in FIG. 2). To this end, the cap 110 also operates, in a known way, as an RNS remover 300. FIG. 3 shows a top plan view of the end of the autoinjector with the cap in place. The autoinjector 100 is in the open state such that the RNS remover and the end of a syringe 200 with an attached RNS 212 are visible. The RNS remover comprises a side wall 302 extending away from the cap, which defines a passageway 304 for receiving the RNS when the cap is fitted to the autoinjector. The front end of the side wall terminates with a gripping member 306. The gripping member is configured to allow easy insertion of the RNS 212 into the passageway whilst preventing its withdrawal thereafter. The RNS can therefore be removed from the syringe as the cap is removed.

In the configuration shown in FIG. 3, the gripping member 306 comprises projections 308 which extend inwardly into the passageway 304, angled away from the front end the side wall 302. As the syringe 200 with RNS is inserted into the slot 112 of the main body 104, the projections 308 are able to flex outwardly, whereas any return motion is prevented by the projections 308 as they come into engagement with the RNS 212.

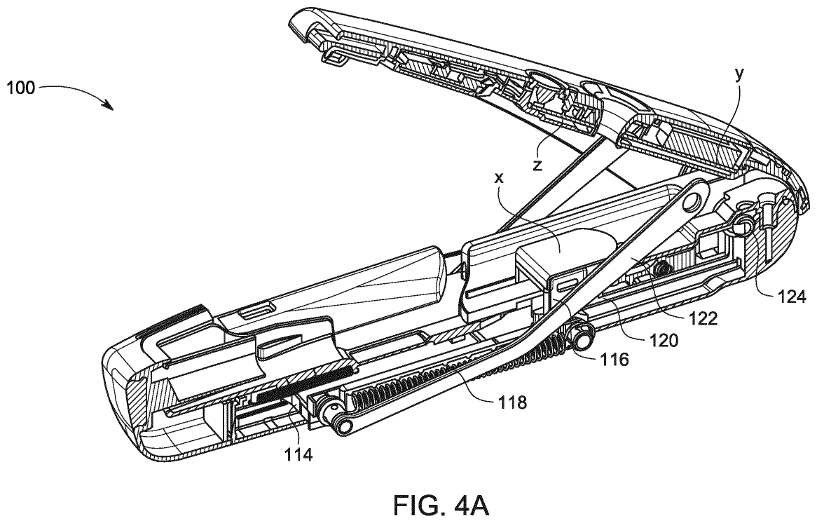

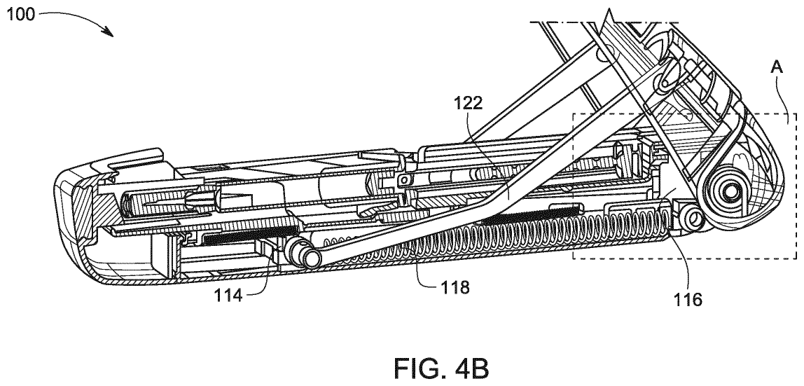

FIGS. 4A and 4B show partial cross-sectional views of the autoinjector 100 during various stages of priming to illustrate the presence and operation of further internal components during lid 106 opening and closing strokes. In particular, it can be seen that the autoinjector 100 comprises a shuttle 114 which is operable to move between a first forward position and a second rearward position along a shuttle guide 120 on the main body 104 of the housing 102, a plunger driver 116 for driving the syringe plunger 204, and a biasing element 118 which couples the shuttle and plunger driver 116. The shuttle and plunger driver are slidably connected to the shuttle guide 120 to permit rearward and forward movement within the housing 102. Unlike the plunger driver, the shuttle is also fixedly connected to the lid 106 via two arm members 122. The plunger driver comprises a rearmost member 117, referred to here as a “push member”, that is located in use behind the rear end of the plunger of an inserted syringe.

The biasing element 118 comprises two extension springs on either side of the device, although only one is visible in the drawings. Prior to any priming, these springs are under slight tension so as to hold the plunger driver 116 and shuttle 114 together. It should therefore be noted that priming, in the context of the extension springs, refers to the process of further tensioning the extension springs into a state whereupon firing can be initiated.

Each of the shuttle guide 120 and the plunger driver 116 comprise part of a latching arrangement, which are configured to cooperate to secure the plunger driver at the rear end of the autoinjector 100. A suitable latching arrangement is described in WO2022179832.

The autoinjector 100 further comprises a torsion spring 124 arranged at the hinged connection between the lid 106 and main body 104 of the autoinjector 100. The torsion spring is coupled to both the lid and main body. In the embodiment shown, one end of the torsion spring is attached to the lid, and the opposing end is attached to the main body of the autoinjector.

Priming of the autoinjector on the lid opening stroke (FIG. 4A) and the lid closing stroke (FIG. 4B) is now described. WO2021058474 describes operation of a similar autoinjector, except that the biasing element described therein further includes a compression spring.

As the lid 106 is opened, the arm members 122 which couple the lid and shuttle 114 together cause the shuttle to move rearwards from the first position to the second position. The shuttle is in constant engagement with the plunger driver 116 so that its rearward travel causes the same rearward travel for the plunger driver. The extension springs coupled between them therefore remains un-primed (i.e. further extended) during lid opening. Near the end of lid opening stroke, the latching arrangement part on the shuttle guide 120 and the plunger driver are brought together such that they are able to cooperate to secure the plunger driver at the rear end of the autoinjector 100.

Lid 106 opening also causes the end of the torsion spring 124 attached to the lid to rotate about its spring axis relative to the opposing end of the torsion spring. This primes the torsion spring on lid opening. When primed, the torsion spring produces a restoring force which tends to urge the lid closed.

Upon closing of the lid 106, whilst the shuttle 114 is free to move forwards along the shuttle guide 120 to the first position, the plunger driver 116 is held at the rear of the autoinjector by the latching arrangement. Thus, during the lid closing stroke, the shuttle and plunger driver separate and the extension springs coupled between them are primed (i.e., further tensioned).

As has already been noted above, the primed torsion spring 124 urges the lid 106 closed. This assists in priming the extension springs 118 during closing, whilst requiring a minimal force to prime the torsion spring during opening. This is important for users of autoinjectors who would otherwise find it difficult to apply the necessary force to close the lid.

Firing of the autoinjector is now described. The firing mechanism is described in more detail in WO2022179832.

To fire the loaded and primed autoinjector, the user urges the front end of the autoinjector 100 into contact with an injection site (e.g., a user's skin). This causes the shroud parts 108a, 108b to move into the retracted position against their biases (e.g., respective springs). As the shroud retracts into the housing 102, the lower shroud 108b permits or causes release of the latching arrangement and the primed extension springs 118a, 118b. The restoring force of the extension springs, acting on the plunger driver 116, drives the plunger driver, and specifically the push member 117, forwards to depress the syringe plunger and force the drug from out of the syringe needle into the injection site.

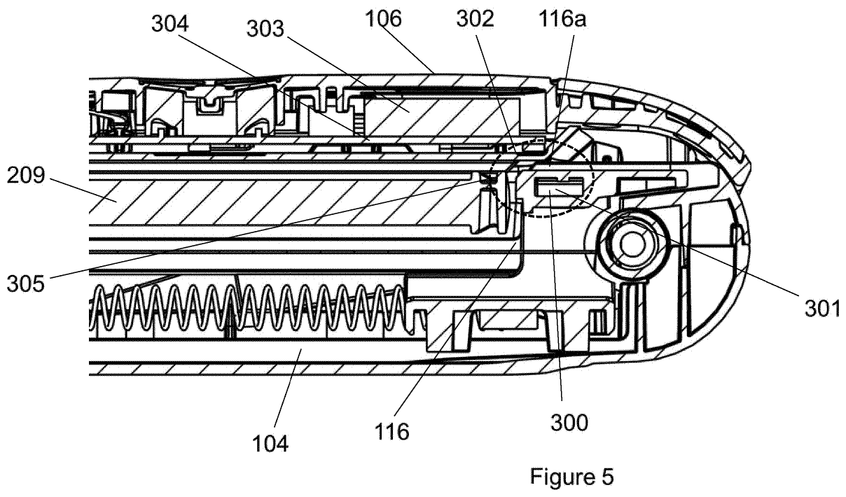

FIG. 5 illustrates a side cross-sectional view of a rear end of the autoinjector 100 as identified by the broken line box A of FIG. 4B (where the cross-section is taken over a generally vertical and axial plane). An upper part 116a of the plunger driver 116 defines a central chamber 300 within which is located a magnet 301. Also visible in FIG. 5 is a Hall effect sensor 302 fixed within the lid 106 of the housing 102. The respective locations of the magnet 301 and the Hall effect sensor 302, x and y respectively, are also pointed out in FIG. 4a, albeit with the plunger driver shown at an intermediate position of its stroke. An electronics module 303 is also provided within the lid 106, electrically coupled to a printed circuit board (PCB) 304, to the underside of which is electrically connected the Hall effect sensor 302. The electronics module comprises a processor, memory, radio transceiver etc, such that it is able to control and monitor signals generated by the Hall effect sensor, process those and any other signals sensed within the autoinjector, control any display features of the autoinjector, and communicate with an external user or other device, e.g. using wireless Bluetooth™ or WiFi interface.

The Hall effect sensor 302 is configured to detect a change of magnetic field within a defined sensing region. This region might have a volume extending beneath the sensor within a range of 5-25 mm², but in any case is sufficiently large for the magnet 301 to be located within the sensing region when the autoinjector is primed and the plunger driver located in its rearmost position (i.e. the position shown in FIGS. 4B and 5). An exemplary sensing region is indicated in FIG. 5 by the broken line 305.

In the primed, pre-firing state, the Hall effect sensor 302 will produce some stable signal, e.g. a fixed or slowly varying DC voltage. Upon firing of the autoinjector, the plunger driver 116, including the upper part 116a and the magnet 301, will move away from the start position and travel forwards through the autoinjector. The movement of the magnet out of the sensing region 305 of the Hall effect sensor 302 will result in a change in the output signal of the sensor. This change is not transient, e.g. a spike as would be seen were a magnet to rapidly enter and leave the sensing region, but permanent, at least until the device is opened, reloaded and re-primed. The operating frequency (sampling rate) of the sensor and/or the electronics module 303 can therefore be relatively low, for example 20 samples per second or less. The only limitation on this frequency is how quickly the user needs to be alerted to the start of an injection. For example, if detection of an initial movement of the plunger driver is used to illuminate a component of a display of the device, the user is unlikely to perceive a delay of 100 ms or less in providing the illumination.

In a similar manner, a second Hall effect sensor may be used to determine when the plunger driver 116 has reached the end of its stroke and is stationary again. FIG. 4A illustrates a position z within the lid 106 at which the second Hall effect sensor may be located. Again, this sensor will have a relatively small sensing region which the magnet 301 will be well outside of when the magnet is in the primed, pre-fired position shown in FIG. 5 and the second Hall effect sensor will produce some steady state electrical output signal to the electronic module 303. After firing, the magnet will travel forward until the plunger driver arrives at its end stop. At this position, the magnet is now within the sensing region of the second Hall effect sensor such that the sensor output will change to a second, steady, state. Once again, the operating frequency/sampling rate at which the sensor is operated and/or its output sampled can be relatively low.

The skilled reader will be able to envisage further embodiments of the invention without departing from the scope of the appended claims. For example, other contactless proximity sensors may be used in place of the Hall effect sensors, e.g. optical, induction, and ultrasound sensors. In place of a magnet or magnetic component, some other detection feature may be used, e.g. a magnetic coil, mirrored surface, etc. It is also possible to swap the locations of the contactless proximity sensor and the detection feature, i.e. fixing the sensor with respect to the plunger driver and the contactless proximity sensor with respect to the main housing.

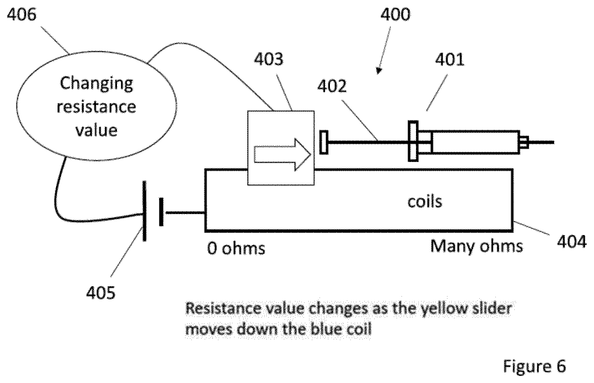

FIG. 6 illustrates schematically an alternative autoinjector configuration 400, but with only relevant parts of the device shown. Also illustrated is a syringe 401 with syringe plunger 402 that is accommodated within a main device housing (not shown). A plunger driver 403 is primed and released to apply a motive force to the syringe plunger. In order to detect motion and position of the plunger driver, a detector comprising a linear reheostat (or “resistance slider”) is provided 404. A series electrical circuit is formed comprising the reheostat 404, a battery 405, and a current detector 406. As the plunger moves up and down the housing, a sliding contact of the rheostat is caused to move up and down a resistance element, e.g. a coil, thereby changing the resistance presented by the rheostat within the electrical circuit. This results in a linear change in the current in the circuit. A measure of the current thereby provides a measure of the position of the sliding contact and therefore the plunger driver.

1. An autoinjector for delivering a dose of medication into a patient from a medication containing syringe or cartridge having a needle affixed thereto, the autoinjector comprising:
a main housing for receiving the syringe or cartridge;
a drive mechanism located substantially within the main housing for providing motive force to deliver medication into the patient from the syringe or cartridge, the drive mechanism comprising a plunger driver axially movable through the housing, between a stationary start position and a stationary end position, to apply said motive force to a plunger of the syringe or cartridge;
a contactless proximity sensor and a detection feature for detection by the contactless proximity sensor;
an electrically powered monitoring unit contained substantially within the main housing and electrically coupled to the contactless proximity sensor;
one of the contactless proximity sensor and the detection feature being fixed with respect to said main housing and the other being fixed with respect to the plunger driver,
wherein, when the plunger driver is in one of the stationary start position and the stationary end position, the detection feature is located at a position within a sensing region of the contactless proximity sensor and, for substantially all of the travel of the plunger driver between the stationary start position and stationary end position, the detection feature is located outside of the sensing region of the contactless proximity sensor.

2. The autoinjector according to claim 1, wherein said drive mechanism comprises one or more springs configured to be primed by user action and which, when primed, generate said motive force.

3. The autoinjector according to claim 1 and comprising a second contactless proximity sensor fixed with respect to the main housing or the plunger driver, wherein, when the plunger driver is in the other of the stationary start position and the stationary end position, the detection feature is located at a position within a sensing region of the second contactless proximity sensor and, for substantially all of the travel of the plunger driver between the stationary start position and stationary end position, the detection feature is located outside of the sensing region of the second contactless proximity sensor.

4. The autoinjector according to claim 1 and comprising a display for displaying an operating state of the autoinjector in dependence upon signals generated by the contactless proximity sensor or sensors.

5. The autoinjector according to claim 1, said main housing comprising a main body and a lid hingedly coupled together, wherein said drive mechanism is mounted to said main body and the or each contactless proximity sensor is fixed to the lid.

6. The autoinjector according to claim 1, the plunger driver comprising a push member configured in use to locate behind a rear end of a plunger of a syringe received within the main housing and to apply said motive force to the plunger, the detection feature being located within said push member.

7. The autoinjector according to claim 1, wherein the or each contactless proximity sensor is a Hall effect sensor and the detection feature is a magnetic or ferromagnetic component.

8. An autoinjector for delivering a dose of medication into a patient from a medication containing syringe or cartridge having a needle affixed thereto, the autoinjector comprising:
a main housing for receiving the syringe or cartridge;
a drive mechanism located substantially within the main housing for providing motive force to deliver medication into the patient from the syringe or cartridge, the drive mechanism comprising a plunger driver axially movable through the housing, between a stationary start position and a stationary end position, to apply said motive force to a plunger of the syringe or cartridge;
a linear rheostat comprising a resistance element and a sliding contact, one of the resistance element and the sliding contact being fixed relative to the main housing and the other being fixed relative to the plunger driver; and a detection circuit comprising a battery and a current detector coupled in series with the rheostat and configured to provide a signal indicative of a resistance presented by the rheostat.

9. The autoinjector according to claim 8, wherein said drive mechanism comprises one or more springs configured to be primed by user action and which, when primed, generate said motive force.

10. The autoinjector according to claim 8 and comprising a display for displaying an operating state of the autoinjector in dependence upon signals generated by the rheostat.

11. The autoinjector according to claim 8, said main housing comprising a main body and a lid hingedly coupled together, wherein said drive mechanism and rheostat is mounted to said main body.

12. The autoinjector according to claim 8, said main housing comprising a main body and a lid hingedly coupled together, wherein said drive mechanism and rheostat is mounted to said main body.
